(19) United States
(12) Patent Application Publication
(10) Pub. No.: US 2026/0256820 A1
(43) Pub. Date: Sep. 3, 2026

(54) AN ANTI-INFECTIVE AGENT, METHOD OF TREATMENT, AND USE THEREOF

(71) Applicant: SASTRA DEEMED UNIVERSITY, THANJAVUR, TAMIL NADU (IN)

(72) Inventors: SAHANA VASUDEVAN, Thanjavur (IN); ADLINE PRINCY SOLOMON, Thanjavur (IN); KARTHI SHANMUGHAM, Thanjavur (IN); DAVID HELMA, Thanjavur (IN)

(21) Appl. No.: 19/114,453

(22) PCT Filed: Sep. 22, 2023
(86) PCT No.: PCT/IN2023/050878

§ 371 Date: Mar. 23, 2025

(30) Foreign Application Priority Data
Sep. 23, 2022 (IN) 202241054663

(51) Int. Cl. A61K 31/7036

(52) U.S. Cl. CPC A61K 31/7036

(57) ABSTRACT

An anti-infective agent for use in treatment of candidiasis is disclosed. A method of treating candidiasis is also disclosed. Said method comprises step of: administering an anti-infective agent, at a dosage that ranges between about 90 nM and about 200 nM. The disclosed anti-infective agent offers at least the advantage of inhibiting Candida species morphogenesis, at very low concentrations.

### FIELD OF THE INVENTION

The present disclosure is generally related to anti-infective agents. Particularly, the present disclosure is related to an anti-infective agent for treating candidiasis. More particularly, the present disclosure is related to: an anti-infective agent for treating candidiasis; method of treating candidiasis; and use thereof.

### BACKGROUND OF THE INVENTION

Candida albicans (C.albicans) is a ubiquitous opportunistic microorganism. It can adhere to various substrates, establish an elaborate biofilm, and invade human tissues, causing superficial and systemic infections, resulting in high mortality rates (about 30% to about 60%).

Currently available treatment options are exceedingly short and mostly limited to three classes: polyenes; azoles; and echinocandins. However, several Candida species have already developed resistance, even to most recently developed anti-infective drugs.

There is, therefore, a need in the art, for: an anti-infective agent for treating candidiasis; method of treating candidiasis; and use thereof, which overcome the aforementioned drawbacks and shortcomings.

### SUMMARY OF THE INVENTION

An anti-infective agent for use in treatment of candidiasis is disclosed. A method of treating candidiasis is also disclosed. Said method comprises step of administering an anti-infective agent. Dosage of said anti-infective agent ranges between about 90 nM and about 200 nM.

The disclosed anti-infective agent offers at least the advantage of inhibiting Candida species morphogenesis, at very low concentrations. Treatments with said anti-infective agent were determined to lead to impaired virulence and reduced yeast-to-hyphal morphogenesis.

### BRIEF DESCRIPTION OF THE DRAWINGS

FIG. 1(a) illustrates anti-biofilm activities of an anti-infective agent, against C.albicans (ATCC SC5314), at varying concentrations, in accordance with various embodiments of the present disclosure;

FIG. 1(b) illustrates anti-biofilm activities of an anti-infective agent, against C.albicans (ATCC 90028), at varying concentrations, in accordance with various embodiments of the present disclosure;

FIG. 1(c) illustrates anti-biofilm activities of an anti-infective agent, against C.albicans (clinical isolate, BF1), at varying concentrations, in accordance with various embodiments of the present disclosure;

FIG. 2(a) illustrates filamentation inhibition activities of an anti-infective agent, at about 200 nM, against C.albicans (ATCC SC5314), after about 24 hours, in accordance with an embodiment of the present disclosure;

FIG. 2(b) illustrates filamentation inhibition activities of an anti-infective agent, at about 90 nM, against C.albicans (ATCC 90028), after about 24 hours, in accordance with an embodiment of the present disclosure;

FIG. 2(c) illustrates filamentation inhibition activities of an anti-infective agent, at about 90 nM, against C.albicans (clinical isolate, BF1), after about 24 hours, in accordance with an embodiment of the present disclosure;

FIG. 3(a) illustrates germ tube inhibition kinetics of an anti-infective agent, at about 200 nM and about 60× magnification, against C.albicans (ATCC SC5314), in accordance with various embodiments of the present disclosure;

FIG. 3(b) illustrates germ tube inhibition kinetics of an anti-infective agent, at about 90 nM and about 60× magnification, against C.albicans (ATCC 90028), in accordance with various embodiments of the present disclosure;

FIG. 3(c) illustrates germ tube inhibition kinetics of an anti-infective agent, at about 90 nM and about 60× magnification, against C.albicans (clinical isolate, BF1), in accordance with various embodiments of the present disclosure;

FIG. 4 illustrates results of qRT-PCR analyses, of biofilm, hyphal, and virulence genes of C.albicans treated with an anti-infective agent, with ITS as a housekeeping gene, in accordance with various embodiments of the present disclosure;

FIG. 5(a) illustrates adhesion and invasion light microscopy images of C.albicans (ATCC SC5314) treated with an anti-infective agent, at about 200 nM and about 40× magnification, in accordance with an embodiment of the present disclosure;

FIG. 5(b) illustrates adhesion and invasion light microscopy images of C.albicans (ATCC 90028) treated with an anti-infective agent, at about 90 nM and about 40× magnification, in accordance with an embodiment of the present disclosure;

FIG. 5(c) illustrates adhesion and invasion light microscopy images of C.albicans (clinical isolate, BF1) treated with an anti-infective agent, at about 90 nM and about 40× magnification, in accordance with an embodiment of the present disclosure;

FIG. 6 illustrates results of homology modelling of C.albicans α-glucosidase, in accordance with an embodiment of the present disclosure; and

FIG. 7 illustrates results of docking studies, of α-glucosidase and an anti-infective agent, in accordance with an embodiment of the present disclosure

### DETAILED DESCRIPTION OF THE INVENTION

Throughout this specification, the use of the words “comprise” and “include”, and variations such as “comprises”, “comprising”, “includes”, and “including”, may imply the inclusion of an element (or elements) not specifically recited. Further, the disclosed embodiments may be embodied, in various other forms, as well.

Throughout this specification, the use of the word “candidiasis” is to be construed as being inclusive of: candidiasis that is caused, by C.albicans.

Throughout this specification, the use of the phrase “Candida species” is to be construed as being inclusive of: C.albicans.

Throughout this specification, the use of the word “treated” is to be construed as: “Candida species that is treated with an anti-infective agent”.

Throughout this specification, the use of the phrase “anti-infective agent” is to be construed as: “acarbose” (C₂₅H₄₃NO₁₈; CAS ID: 56180-94-0).

Throughout this specification, the disclosure of a range is to be construed as being inclusive of: the lower limit of the range; and the upper limit of the range.

Throughout this specification, the words “the” and “said” are used interchangeably.

Also, it is to be noted that embodiments may be described as a method. Although the operations in a method are described as a sequential process, many of the operations may be performed in parallel, concurrently, or simultaneously. In addition, the order of the operations may be re-arranged. A method may be terminated, when its operations are completed, but may also have additional steps.

An anti-infective agent for treating candidiasis and method of treating candidiasis are disclosed. Surprisingly, the Inventors have determined that an anti-diabetic agent can be used as an anti-infective agent for treating candidiasis. The disclosed anti-infective agent was tested, as follows:

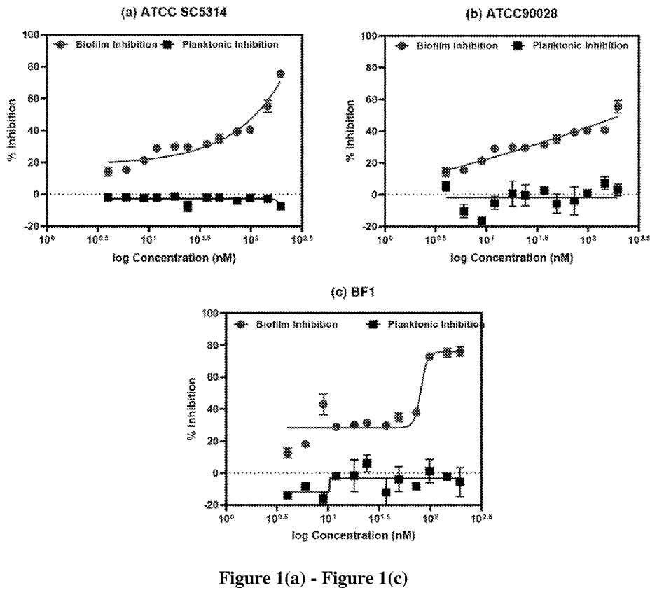

Cultures of C.albicans strains (ATCC SC5314, ATCC 90028, and clinical isolate, BF1) were maintained, in YPD (Yeast Extract, Peptone, and Dextrose) agar, at about 37 degrees Centigrade. As illustrated, in FIG. 1(a), FIG. 1(b), and FIG. 1(c), biofilms were inhibited, by the anti-infective agent, in a strain-specific manner. Effective inhibition concentrations were determined to be between about 90 nM and about 200 nM.

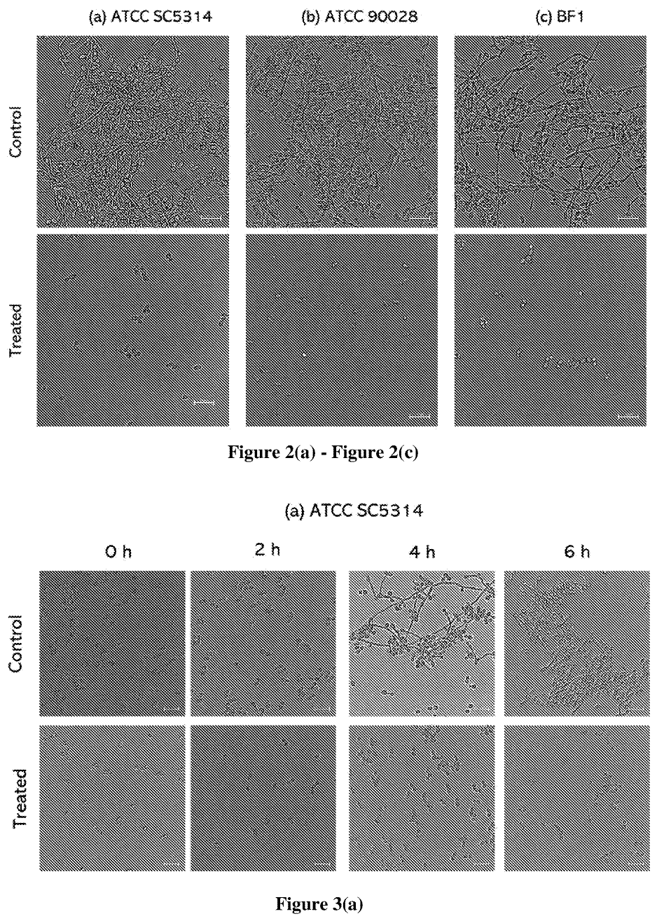

As illustrated, in FIG. 2(a), FIG. 2(b), and FIG. 2(c), treatments with the anti-infective agent, at nanomolar concentrations, were determined to visibly impair filamentation, in presence of hyphal-inducing media.

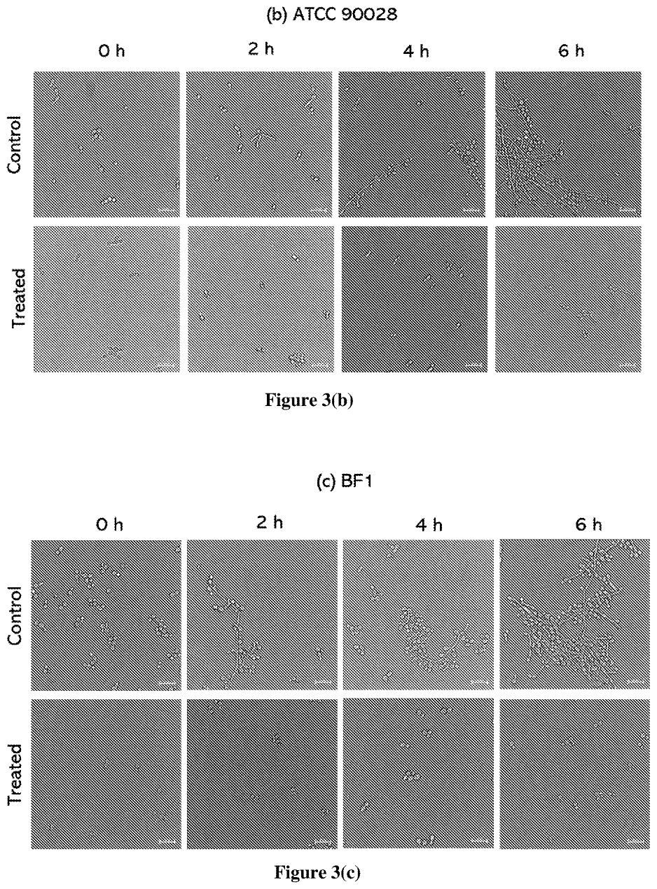

As illustrated, in FIG. 3(a), FIG. 3(b), and FIG. 3(c), untreated cells showed germ tube development, from yeast cells, to a well-established hyphal network. The treatments with the anti-infective agent, on the other hand, were determined to inhibit morphogenic transformations, from yeast.

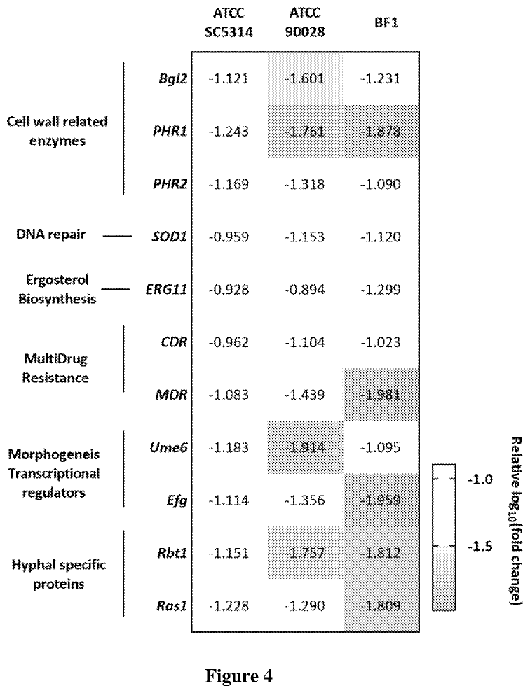

As illustrated, in FIG. 4, the clinical isolate, BF1, was determined to be more sensitive to the treatment with the anti-infective agent. Data were obtained, in triplicates, and analyses were performed, by the 2^{-ΔΔCt} method.

Nevertheless, all considered genes were determined to be downregulated. BGL2, PHR1 and PHR2 are genes responsible for cell wall structures and association with other interspecies interactions. Ume6 and Efg are transcriptional regulators responsible for morphogenic shifts.

Rbt1 and Ras1 are hyphal-specific proteins, while CDR and MDR are genes responsible for anti-microbial resistance. Genes Erg1 and SOD1 are critical for virulence. Thus, the treatments with the anti-infective agent were determined to lead to impaired virulence and reduced yeast-to-hyphal morphogenesis.

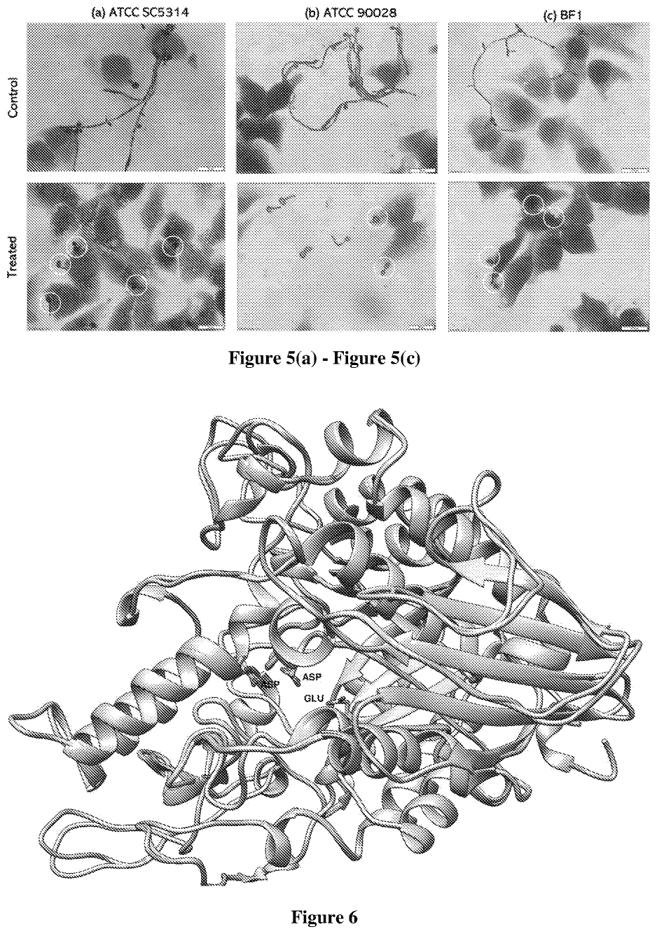

As illustrated, in FIG. 5(a), FIG. 5(b), and FIG. 5(c), the treatments with the anti-infective agent were determined to lead to shorter germ tube formations (reduced adhered and invaded fungal cells).

Crystal structure of C.albicans is not experimentally solved. Hence, homology modelling was performed, by using isomaltose of Saccharomyces cerevisiae (PDB ID: 3AXI). Sequence similarity of about 48.71% was achieved, between the S. cerevisiae isomaltose and C.albicans α-glucosidase, whereas about 99% query coverage was achieved.

As illustrated, in FIG. 6, catalytic sites (ASP, ASP, and GLU) were determined to be conserved. Root Mean Square Deviation value was determined to be only about 0.286 Å. Blue colour represents the S.cerevisiae isomaltose, while sandal colour represents modelled protein of C.albicans α-glucosidase.

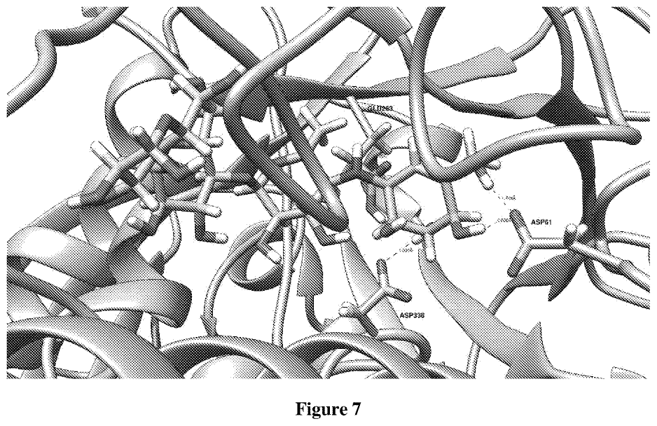

As illustrated, in FIG. 7, the anti-infective agent was determined to make stable interactions with catalytic residues ASP61, GLU263, and ASP338.

The disclosed anti-infective agent offers at least the advantage of inhibiting C.albicans morphogenesis (or Candida species morphogenesis), at very low concentrations.

It will be apparent to a person skilled in the art that the above description is for illustrative purposes only and should not be considered as limiting. Various modifications, additions, alterations, and improvements, without deviating from the spirit and the scope of the disclosure, may be made, by a person skilled in the art. Such modifications, additions, alterations, and improvements should be construed as being within the scope of this disclosure.

We claim:

1. Acarbose for use in treatment of candidiasis.

2. The acarbose for use in treatment of candidiasis, as claimed in claim 1, wherein: said candidiasis is caused, by C.albicans.

3. A method of treating candidiasis, comprising step of: administering acarbose, at a dosage that ranges between 90 nM and 200 nM.

4. The method of treating candidiasis, as claimed in claim 3, wherein: said candidiasis is caused, by C.albicans.

5. The method of treating candidiasis, as claimed in claim 3, wherein: said dosage is 90 nM.

6. The method of treating candidiasis, as claimed in claim 3, wherein: said dosage is 200 nM.
